(19) United States
(12) Patent Application Publication
(10) Pub. No.: US 2021/0106225 A1
(43) Pub. Date: Apr. 15, 2021

(54) MULTIFUNCTIONAL OPHTHALMIC DEVICE

(71) Applicant: Vision for Mars Technologies LLC, South Jordan, UT (US)

(72) Inventors: Neil Griffin, Melbourn (GB); Roger Clarke, Melbourn (GB)

(73) Assignee: Vision for Mars Technologies LLC, South Jordan, UT (US)

(21) Appl. No.: 16/887,440

(22) Filed: May 29, 2020

(30) Foreign Application Priority Data
Oct. 11, 2019 (GB) 1914750.3

(51) Int. Cl. A61B 3/18; A61B 3/10; A61B 3/12; A61B 3/14; A61B 3/024; A61B 3/00

(52) U.S. Cl. CPC A61B 3/18; A61B 3/1015; A61B 3/102; A61B 3/12; A61B 3/185; A61B 3/024; A61B 3/0041; A61B 3/0091; A61B 3/14

(57) ABSTRACT

Embodiments of the present disclosure are directed to ophthalmic system including a first eyepiece configured to define a first subject region within a field of view of the first eyepiece, the first subject region being configured such that light may travel between the first subject region and one or more first ophthalmic devices through the first eyepiece, and a second eyepiece configured to define a second subject region within a field of view of the second eyepiece, the second subject region being configured such that light may travel between the second subject region and one or more second ophthalmic devices through the second eyepiece, wherein the second subject region extends across a greater angle in the field of view of the second eyepiece than does the first subject region in the field of view of the first eyepiece.

### GOVERNMENT SUPPORT

This invention was made with government support under Federal Awarded No. NNX16A069A awarded by the National Aeronautics and Space Administration. The government has certain rights in the invention.

### CROSS-REFERENCE TO RELATED APPLICATIONS

The present application claims priority to GB Patent Application No.1914750.3, filed Oct. 11, 2019, the entirety of which is hereby incorporated by reference.

### FIELD

The present disclosure relates to an ophthalmic system for providing measurements relating to plural characteristics of a user's eye.

### BACKGROUND

Many methods have been developed in the field of ophthalmology for studying the structure and condition of the human eye. Modern ophthalmic practices make extensive use of complex optical devices to image and measure the eye, examples of which include optical coherence tomography (OCT) devices, used to obtain detailed images of the retina and anterior segment of the eye; Shack-Hartman wavefront sensors, used to measure aberrations in the refractive structures of the eye such as the lens; retinal cameras, which are used to produce photographs of the retina; corneal topography devices, which provide measurements of the surface of the cornea; and perimetry devices, which can perform partially or wholly automated tests for identifying and measuring defects in the visual field of a patient.

Ophthalmic devices such as those listed above each require specific combinations of optical apparatus, for example light sources for illuminating the retina or other structures in the eye; lenses and mirrors, some or all of which may be adjustable (e.g. to control the focus and direction of light illuminating the eye or reflected from it); and cameras, photodiodes and other types of detector. These devices may also incorporate apparatus for producing a display that is perceptible to the patient who is the subject of the test, for example in the form of a target for focussing the patient's gaze on a fixed point in space.

Owing to the complex nature of these arrangements of optical apparatus, ophthalmic devices are typically large and difficult to transport. This means that providing a comprehensive range of ophthalmic tests at one site can require a substantial amount of space, and deploying the apparatus required to do so can be arduous. Deploying ophthalmic devices rapidly (as may be required in, for example, disaster zones) and/or in environments with limited space or infrastructure, for example small clinics, remote sites and aerospace and maritime settings, is impractical.

There is hence a need for a compact, easily transportable system capable of performing different ophthalmic tests.

### SUMMARY

A first aspect of the present disclosure provides an ophthalmic system for providing measurements relating to plural characteristics of a user's eye processed by a common processing device, the system comprising: a first eyepiece configured to define a first subject region within a field of view of the first eyepiece, the first subject region being configured such that light may travel between the first subject region and one or more first ophthalmic devices through the first eyepiece, each first ophthalmic device being configured to receive light from the first subject region and/or output light to be directed to the first subject region, wherein the first eyepiece and the one or more first ophthalmic devices are arranged such that the light received and/or output by each first ophthalmic device travels between the respective first ophthalmic device and the first eyepiece along a first common path defined by one or more first optical components; a second eyepiece configured to define a second subject region within a field of view of the second eyepiece, the second subject region being configured such that light may travel between the second subject region and one or more second ophthalmic devices through the second eyepiece, each second ophthalmic device being configured to receive light from the second subject region and/or output light to be directed to the second subject region, wherein the second eyepiece and the one or more second ophthalmic devices are arranged such that the light received and/or output by each second ophthalmic device travels between the respective second ophthalmic device and the second eyepiece along a second common path defined by one or more second optical components; wherein the first and second eyepieces are configured such that light can be directed to and received from the second subject region across a range of angles having a greater magnitude than the range of angles across which light can be directed to and received from the first subject region by the first eyepiece, and wherein each of the first and second ophthalmic devices is in communication with the common processing device so as to be controllable by the common processing device and/or output measurements to the common processing device.

The existence of a “common path” as defined above means that light travelling between one of the first and second eyepieces and any one of its respective ophthalmic devices will travel along a finite region of the ophthalmic system that is also traversed by light travelling between the eyepiece and each of the other respective ophthalmic devices. The each common path could span the distance between the respective eyepiece and an optical component, for example, and/or between two or more such optical components. It should be understood that light travelling between each ophthalmic device and the respective eyepiece may leave, join or rejoin the associated common path. For example, light travelling between each ophthalmic device and its respective eyepiece may be separated from the respective common path at a different point to light travelling between the eyepiece and other ophthalmic devices.

The “optical components” defined above could be provided by any structure that is capable of interacting with light so as to alter its direction, focus or any other geometric property, e.g. by reflection or refraction.

Each “subject region” is a region in which the user's eye is intended to be positioned while he interacts with the ophthalmic device(s) via the relevant eyepiece. The extent and position of each subject region are defined by the structure and optical properties of the eyepiece: the subject region is the entire region from within which light can be received from and directed to the respective ophthalmic devices via the eyepiece in the manner defined above. Each of the first and second eyepieces will be capable of directing light to and receiving light from the respective subject across a respective range of angles. That is to say that for each eyepiece there will be a greatest possible angle that can exist between any two rays of light travelling between the subject area and its respective ophthalmic device(s), and any pair of rays having a greater angle between them will not both be corrected conveyed between the subject area and the ophthalmic device(s). This greatest possible angle corresponds to the magnitude of the range of angles defined above. The magnitude of the range of angles and the configuration of the subject area of each eyepiece may depend on, inter alia, the optical power of the respective eyepiece and its physical dimensions.

In preferred embodiments the first and second eyepieces are configured such that the first and second subject regions do not intersect or overlap one another. It is nonetheless possible for the first and second eyepieces to be arranged such that their respective subject areas do overlap and/or intersect, but the each of the first and second eyepieces should not be arranged so as to receive light from or direct light to the second or first ophthalmic devices (i.e. the ophthalmic devices associated with the other eyepiece) respectively.

Preferably each first and/or second optical component comprises one or more mirrors and/or lenses arranged to direct light along the first or second optical path respectively. The first and/or second optical components could also include, however, other structures such as optical fibres, prisms or apertures.

In some embodiments the one or more first ophthalmic devices comprise one or more of an optical coherence tomography device, a retinal camera and a wavefront sensor, and most preferably include each of these. Preferably the one or more second ophthalmic devices comprise a visual field test tool, such as an Amsler grid or a display suitable for performing a Humphrey perimetry test. The first and second ophthalmic devices may also include other types of ophthalmic device not listed here.

In some embodiments the one or more first ophthalmic devices and/or the one or more second ophthalmic devices comprise a fixation display configured to produce a target visually perceptible from the subject region of the respective eyepiece such that a user at the respective subject region may focus his vision on the target. This enables the user to focus keep his vision focused along a particular direction during a test performed using any of the ophthalmic devices included in the system, which is desirable in many ophthalmic tests. The fixation display could, for example, present a target in the form of a compact, luminous feature perceptible when the user looks towards the relevant eyepiece from within the respective subject region.

In preferred embodiments the first and second eyepieces, each of the first and second ophthalmic devices and the processing device are contained by a common housing. This improves the compactness of the system, though in other embodiments one or more of these elements could be outside of the housing. For example, the first and second ophthalmic devices could be in communication with the processing device wirelessly, and the processing device could be located elsewhere.

In some embodiments, the system comprises a first enclosure configured to enclose the first common path and/or one or more of the first ophthalmic devices; and/or a second enclosure configured to enclose the second common path and/or one or more second ophthalmic devices; and most preferably comprises both. Such an enclosure can reduce contamination of the obtained measurements due to light from outside of the system, and also prevent light from outside the enclosure being directed towards the respective subject area in such a way that distracts the user during a test or otherwise interferes with his ability to complete the procedure. A further particular advantage of the enclosure is that it may be configured to optically isolate the set of first ophthalmic device and the set of second ophthalmic devices from one another. This can prevent light produced by the first ophthalmic devices from reaching the second ophthalmic device and the second eyepiece, and vice versa, and can allow the first and second ophthalmic devices to be arranged in a compact configuration without light produced by either set contaminating light produced by and/or directed to the ophthalmic devices in the other set. Hence, in best preferred implementations, one or both of the first enclosure and the second enclosure is configured so as to prevent light not transmitted through the respective first or second eyepiece from entering the respective first or second common path.

### LIST OF FIGURES

Examples of an ophthalmic system will now be described with reference to the accompanying drawings, in which:

FIG. 1 shows schematically a first eyepiece and associated ophthalmic devices suitable for incorporation in an ophthalmic system, according to one or more embodiments shown and described herein;

FIG. 2 shows schematically a second eyepiece and a second ophthalmic device suitable for incorporating in an ophthalmic system, according to one or more embodiments shown and described herein;

FIG. 3 shows detailed examples of (a) a first eyepiece and (b) a second eyepiece each suitable for incorporating in an ophthalmic system, according to one or more embodiments shown and described herein; and

FIG. 4 shows (a) an isometric view and (b) to (d) orthographic views of an ophthalmic system, according to one or more embodiments shown and described herein.

### DETAILED DESCRIPTION

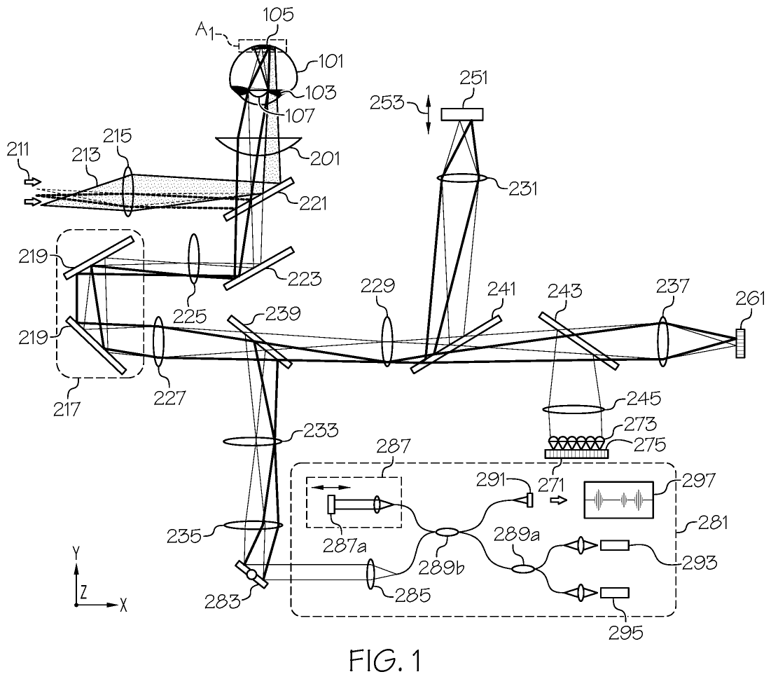

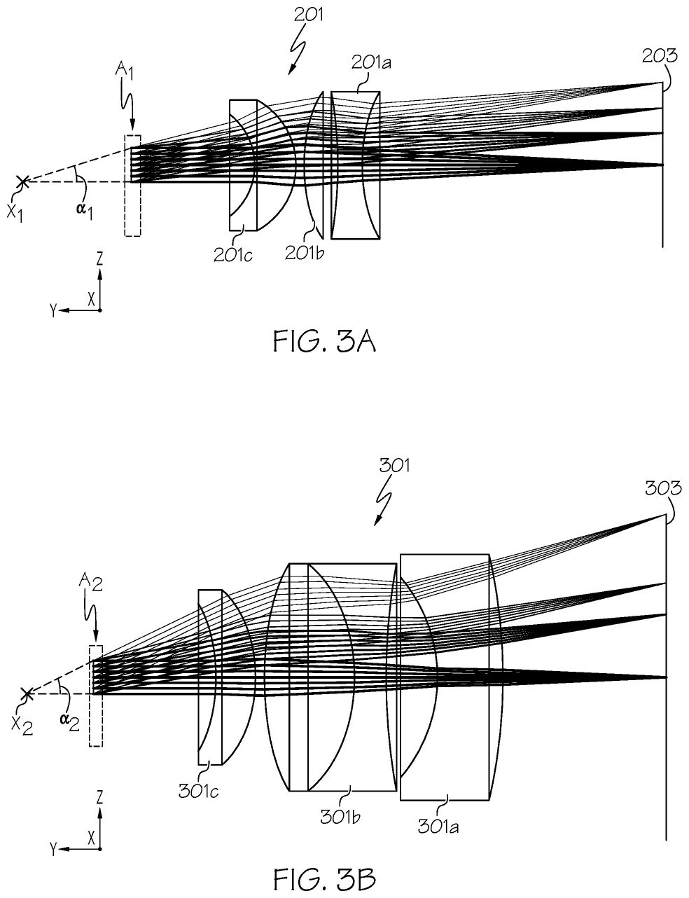

FIG. 1 shows schematically a first eyepiece 201 and a plurality of first ophthalmic devices in an arrangement suitable for implementation in an ophthalmic system in accordance various embodiments provided herein. In this drawing the first eyepiece 201 is shown as a simple convex lens, though a detailed example of another suitable first eyepiece will be described later with reference to FIG. 3(a). The first eyepiece 201 is configured to receive light from a plurality of first ophthalmic devices and direct the received light towards a first subject region A₁, and to receive light from within the first subject region A₁ and direct the received light towards the first ophthalmic devices (along a first optical path defined by a series mirrors and lenses which will be described below). In this example the first ophthalmic devices may include an illumination light 211, a fixation display 251, a retinal camera 261, a wavefront sensor 271 and an optical coherence tomography (OCT) device 281. Each of these devices will be described in detail below.

Positioned within the subject region A₁ of the first eyepiece is an eye 101 of a patient. The patient is positioned such that the pupil 103 of the eye 101 faces the first eyepiece 201.

The retinal illumination light 211 is arranged to produce light 213 directed towards a lens 215, which focusses the produced light onto a partial mirror 221. The partial mirror 221 is configured to reflect the light 213 towards the first eyepiece 201 along a part of a first common path of the first eyepiece 201 (the first common path will be further discussed later), which itself focusses the light 213 through the pupil 103 and onto the retina 105 of the eye 101. The light reflected by the retina 105 may be measured by the various first ophthalmic devices associated with the first eyepiece 201. In some embodiments the retinal illumination light 211 produces light 213 in the shape of a ring such that the light 213 is not incident on the centre of the pupil 103 and only on its peripheral areas. This prevents light being reflected by the pupil 103 itself (rather than the retina 105) through the first eyepiece 201 and towards the first ophthalmic devices (which is advantageous as any such reflection may interfere with measurements produced by the first ophthalmic devices). The retinal illumination light 211 is, in this example, arranged to be near the first eyepiece 201 in order to minimise the number of optical components (e.g. mirrors and lenses) with which the light 213 output by it must interact (as shown in FIG. 1, the light 213 only interacts with the first partial mirror 221 before being received by the first eyepiece 201). This reduces the amount of the light 213 that is reflected towards the first optical devices as a result of interactions with the optical components and hence mitigates the effect of any such reflected light on the obtained measurements. In embodiments, the light 213 output by the retinal illumination light 211 is in the near infra-red portion of the spectrum (or some other non-visible portion of the spectrum) such that the light 213 is not perceptible to the patient.

The first partial mirror 221 is configured to permit the transmission of at least some of the light reflected by the retina 105 therethrough towards another mirror 223 (which in the arrangement shown is preferably a full mirror). The mirror 223 directs the light towards a first field lens 225, which focusses the reflected light towards a focus adjustment module 217. The focus adjustment module 217 comprises a pair of mirrors 219 which can together be moved towards or away from the first field lens 225 along at least the X axis (e.g. by a motor) in order to control the focus of light transmitted between the first eyepiece 201 and the first ophthalmic devices. The focussing module 217 may also be controlled in order to compensate for spherical aberrations produced by the lens 107 of the patient's eye, and/or aberrations produced by the optical components in the system (e.g. the first field lens 225 and the mirror 223) that are included in the first common path of the first eyepiece 201. The light directed into the focussing module 217 is reflected by each mirror 219 in turn so as to be directed towards a second field lens 227. The second field lens 227 in turn focusses the light from the focussing module 217 towards a second partial mirror 239, which permits some of the light directed towards it by the second field lens 227 to be transmitted towards a third field lens 229, which directs the light onto a third partial mirror 241.

The third partial mirror 241 permits some of the light incident on it to be transmitted therethrough towards a fourth partial mirror 243, which permits a fraction of the light to be transmitted towards the retinal camera 261 and the remaining fraction to be reflected towards the wavefront sensor 271. The fourth partial mirror may in some examples prevent the transmission of wavelengths not suitable for being recorded by the retinal camera 261, for example.

A focussing lens 237 focusses the light that is transmitted through the fourth partial mirror 243 onto the retinal camera 261. The retinal camera 261 may thus record an image of the retina 105 that is produced by the focussing lens 237 from the light output by the retinal illumination light 211 and reflected by the retina 105. The retinal camera may be a digital camera adapted to record the wavelength(s) produced by the retinal illumination light 211 (for example one that comprises a charge couple device (CCD) defining an array of pixels each responsive to the light output by the retinal illumination light 211).

The light reflected by the fourth partial mirror 243 is received by a collimating lens 245, which is configured to collimate the light (partially or exactly) along the Y axis and direct it towards the wavefront sensor 271. The wavefront sensor 271 in this example is a Shack-Hartmann wavefront sensor and comprises an array of lenslets 273 arranged to focus the collimated light onto a detector 275. The lenslets 273 can each be individually tilted with respect to the plane of the X and Z axes in order to correct for aberrations in the received lights, and measuring the configuration of the lenslets 273 required to focus the light at each point on the detector can yield measurements of aberrations introduced to the light by the lens 107 of the patient's eye 101.

The third partial mirror 241, which was introduced above, is arranged to reflect (at least some, preferably all of) the light produced by the fixation display 251 into the first common path towards the first eyepiece 201. The fixation display 251 is configured to produce light so as to present a target perceptible to the patient in the subject region A₁. The target may be in the form of a small, bright feature or a pattern of such features, for example. The light output by the fixation display 251 is directed towards the third partial mirror 241 and reflected into the same section of the first common path which light to be recorded by the retinal camera 261 and the wavefront sensor 271 travels. The light produced by the fixation display 251 travels to the first eyepiece via the third field lens 229, second partial mirror 239 (through which at least some of it is transmitted), second field lens 227, mirrors 219 of the focussing module 217, first field lens 225, mirror 223 and first partial mirror 221. The light produced by the fixation display 251 is then directed by the first eyepiece 201 into the first subject region A₁, where it is received by the eye 101 of the patient. The fixation target 251 can assist the patient in keeping the eye 101 focused along a particular direction (e.g. one most suitable for imaging the retina using the retinal camera 261, which could be the direction directly towards the centre of the first eyepiece 201). The fixation target 251 is, in this example, movable (e.g. by a motor) along the Y axis (as indicated by the arrows 253), which may, for example, allow the depth of the patient's focus to be varied (which may for example be useful in measuring aberrations of the eye's lens 107 while shaped by the eye 101 to different focal lengths). The appearance of the target itself may also be modified, e.g. by displaying different images on the fixation target 251.

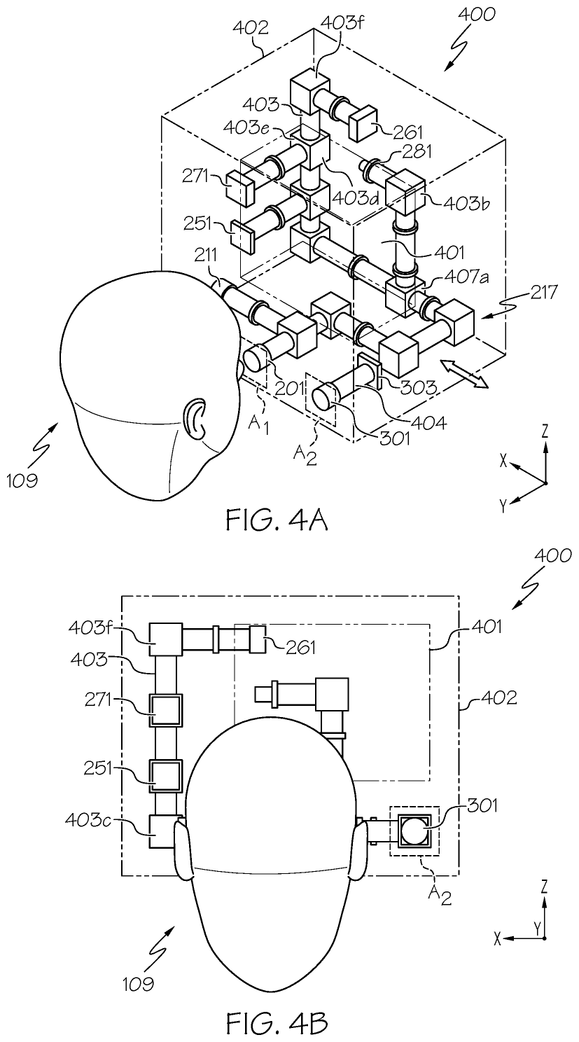

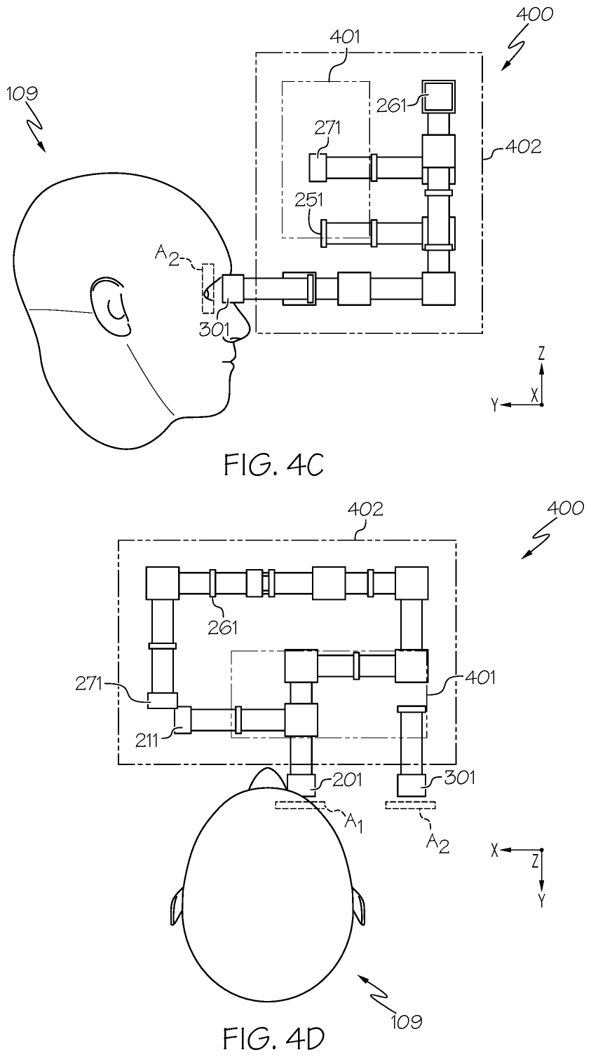

The OCT device 281 measures the topography of the retina 105 by interferometry, i.e. recording variations in the path length of a beam of light reflected from the retina in relation to that of a beam of light directed along a reference path by measuring the interference of light from the two paths. A light source in the form of a super-luminescent diode 293 generates the light to be used to perform the OCT measurements. (In this example the light source is a super-luminescent photodiode but could alternatively be, for example, a laser.) The light output by the diode 293 is coupled to an optical fibre. Some of the light is directed into a reference arm 287 adjustable (by the movement of a mirror 287a in the reference arm, moveable along the X axis as indicated by the arrow shown) so as to vary the length of the reference path, and some is directed towards a lens 285 which directs the light towards a galvanometer mirror 283. The mirror galvanometer 283 comprises a mirror that reflects the light produced by the super-luminescent photodiode 293 and is mechanically coupled to a galvanometer such that in response to a current passing through the galvanometer, the mirror is deflected. This allows the position of the mirror to be finely controlled so as to vary the position at which the light from the diode 293 is incident on the retina 105. Once reflected by the mirror galvanometer 283, the light from the diode passes through a lens 235 and a fourth field lens 233, is at least partially reflected by the second partial mirror 239 through the second field lens 227 into the focussing module 217, successively reflected by both mirrors 219 towards the first field lens 225, which directs it onto the mirror 233 whereby it is directed through the first partial mirror 221 and into the first eyepiece 201. The first eyepiece 201 directs the light into the first subject region A₁, and by controlling the mirror galvanometer 283 the light can be directed onto the desired part of the retina 105. The light incident on the retina is reflected back towards the OCT device via the same set of lenses and mirrors, and is reflected by the mirror galvanometer 283 towards the lens 285, which directs the light back into the optical fibre. The light received from the retina 105 and light from the reference arm are coupled by a fibre coupler 289b, which outputs the combined light towards a detector 291, which records the intensity of the light that it receives. The intensity of the light received by the detector 291 depends on the relative phases, and hence the difference between the path lengths, of the light from the retina and that from the reference arm 287. The detector outputs data 297 pertaining to the measured intensity, which can be analyzed to infer the topography of the retina. The data 297 may be received by a common processing device, an example of which will be described later with reference to FIGS. 4(a) to 4(d). The OCT device 281 may also be controlled by such a processing device (e.g. to control the position of the mirror 281a and/or the orientation of the mirror galvanometer 283).

The first ophthalmic devices shown in FIG. 1 (the retinal illumination light 211, the fixation display 251, the wavefront sensor 271, the retinal camera 261 and the OCT device 281) each direct light towards and/or receive light from the first eyepiece 201 that has travelled along the first common path, which is defined by a plurality of optical components, namely the first partial mirror 221, the mirror 223, the first field lens 225, the mirrors 219 of the focussing module 217, the second field lens 227, the second partial mirror 239, the third field lens 229, the third partial mirror 241 and fourth partial mirror 243.

In this schematic example the optical components and the first ophthalmic devices associated with the first eyepiece 201 are shown as being arranged in a common plane perpendicular to the Z axis. As will be discussed later, however, the components may be arranged in three dimensions. For example, the third partial mirror 241 could be rotated about the X axis in order to allow to fixation display 251 to be positioned directly above or below it along the Z axis. Similarly, the second partial mirror 239 may be rotated about the X axis to allow the OCT device to be arranged above or below the other components along the Z axis. Rotating the mirror galvanometer 283 about the Y axis would allow the orientation of the OCT device 281 about the Y axis to be changed.

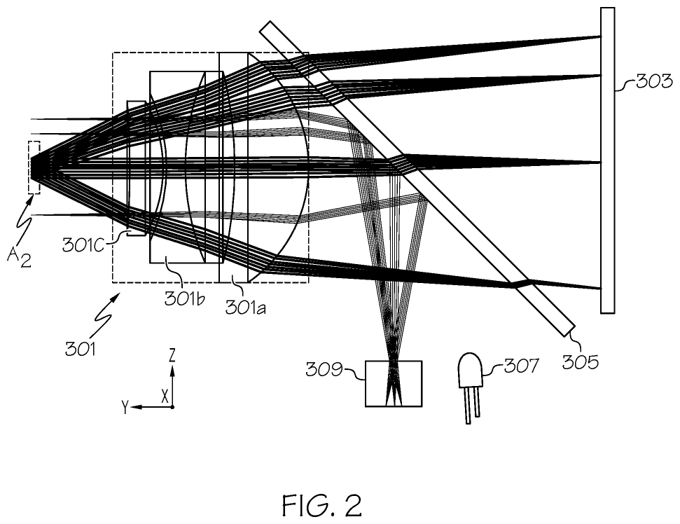

FIG. 2 shows an example of a second eyepiece 301, which is configured to receive light from a second ophthalmic device 303 and direct the received light towards a second subject region A₂.

The second eyepiece 301 comprises three lenses. A first lens 301a has one convex face (on the side facing the second ophthalmic device 303) and one concave face (on the opposed side). A second lens 301b has two concave faces, one on either side of the second lens 301b. A third lens 301c has one convex face (on the side facing the second ophthalmic device 303) and, on the opposite side, a concave face (although the radius of curvature of this face is greater than that of the concave faces of the first and second lenses 301a, 301b). The second eyepiece 301 could, however, be provided by any eyepiece suitable of receiving light from one or more second ophthalmic devices and directing the received light across a second subject A₂ from within which light can be directed to the second ophthalmic device 303 and/or received from the second ophthalmic device 303 across a greater range of angles than light can be directed between the first subject region A₁ of the first eyepiece 201 described above and its respective first ophthalmic devices.

In this example the second ophthalmic device 303 is a visual field test tool 303, from which the second eyepiece 301 is configured to receive light to be output to the second subject region A₂. The visual field test tool could be, for example, an Amsler grid, i.e. a grid of horizontal and vertical lines used to identify defects or aberrations in the field of view of the patient, or any other device suitable for performing a visual field test (e.g. a display suitable for carrying out a Humphrey perimetry test). The Amsler grid could, for example, be projected onto a screen or other such display provided as part of the visual field test tool 303, produced by an electrical or electronic screen (e.g. a cathode ray tube screen, an LCD screen, plasma screen or organic LED screen), or printed, painted or otherwise disposed on such a display and illuminated by a source of visible light (not shown) so as to be perceptible to the patient when viewed through the second eyepiece 301. In this example the light directed from the visual field test tool passes through a partial mirror 305, which is arranged between the visual field test tool 303 and configured to permit at least some of the light from the visual field test tool incident on it to pass through to the eyepiece 301, whereby the received light is directed towards the subject region A₂. The visual field test tool 303 could also incorporate a feedback device for receiving feedback from the patient during a visual field test, for example a button that the patient is instructed to press under certain conditions (e.g. in response to the presentation of visual stimuli during a Humphrey perimetry test).

In this example a pupil tracking camera 309 (which is another second ophthalmic device) is arranged to monitor the position of the position of the pupil of a patient in the subject region A2 by detecting light that is reflected by the eye, through the second eyepiece 301 and reflected by the partial mirror 305. There is also provided an illuminating source 307 which produces radiation detectable by the camera 309. The radiation produced is directed towards the partial mirror so as to be reflected into the eyepiece, whereby it is directed towards the second subject region A₂. The radiation may then be reflected by the eye of a patient in the second subject region A₂, directed by the eyepiece 301 onto the partial mirror 305 and reflected towards the camera 309. The camera 309 is configured to detect the radiation (as was explained above) and the position of the pupil can be monitored based on the received image. This information can be used to ensure that the vision of the patient is focused in the appropriate direction during a visual field test performed using the visual field test tool 303 (since the position and orientation of the pupil will be determined by where the patient's vision is focused). The mirror in this example defines a second common path along which light may travel between the second eyepiece 301 and the second ophthalmic devices described, i.e. the camera 309, illuminating source 307 and the visual field test tool 303.

FIG. 3(a) shows an example of the interior construction of a first eyepiece 201 suitable for implementing the ophthalmic system described above. The first eyepiece 201 includes a first lens 201a having two concave faces, a second lens 201b having a planar face on the side of the first lens 201a and a convex face on the opposing side, and a third lens 201c, which has one convex face and one concave face. In this example the first eyepiece is shown as receiving light from an exemplary source 203, which is focused by each of the first, second and third lenses 201a, 201b, 201c in turn and directed into a first subject region A₁. The light output by the first eyepiece 201 converges towards a first position X₁ at an angle α₁ to the direction along which the first eyepiece 201 is aligned (in this example the Y axis). The first eyepiece 201 is thus configured so as to direct light into and receive light from the first subject region across a range of angles 2α₁.

FIG. 3(b) shows a detailed view of the second eyepiece described above with reference to FIG. 2. In this example light from an exemplary source 303 is received by the convex side of the first lens 301a, focused by the first, second and third lenses 301a, 301b, 301c in turn and output at the convex face of the third lens 301c into the subject region A₂ and towards a second position X₂ at an angle α₂ to the direction along which the second eyepiece 301 is aligned (again the Y axis in this example), which is greater than the angle α₁ described above. The second eyepiece 301 is thus configured so as to direct light into and receive light from the second subject region across a range of angles 2α₂.

FIG. 4(a) shows an isometric view of an embodiment of an ophthalmic system 400. The first eyepiece 201 and the second eyepiece 301 are arranged so as to be accessible to a patient 109 whose eye(s) is or are to be measured using the ophthalmic system 400, and are disposed at the side of a housing 402. The housing 402 contains all of the first and second ophthalmic devices associated with each respective eyepiece. In this example the first common path along which light travels between the first eyepiece 201 the first ophthalmic devices is arranged inside a first enclosure 403, which is formed by a framework of opaque, hollow tubes and junctions inside of which the light can travel along the first optical pathway. The first enclosure 403 can include all of the optical components (i.e. the various mirrors, partial mirrors and lenses) defining the first common path described above with reference to FIG. 1. Similarly, the second eyepiece 301 in this example is configured to transmit light from the second subject region A₂ into a second enclosure 404 containing the visual field test tool 303 and the partial mirror 305 (and the illuminating source 207 and pupil tracking camera 309, if provided). Each of the first and second enclosures 403, 404 in this example is formed of a material that is substantially opaque to visible light so as to prevent light not intended to be directed to or output by one of the first or second ophthalmic devices entering the respective common path. In some embodiments, there may not be any enclosure. For example, all of the first and second ophthalmic devices and their associated optical components could be arranged inside a common housing (e.g. the housing 402) without being separated by any enclosure or other such structure.

As was explained above, the arrangement of first ophthalmic devices shown in FIG. 1 may be reconfigured in three dimensions by orienting the mirrors and partial mirrors that it contains appropriately. This is the case in the exemplary system 400 shown in FIG. 4. The OCT device 281 is positioned above the plane in which the focussing module 217 and the first eyepiece 201 are arranged, for example. The second partial mirror 239 shown in FIG. 1 could be arranged at a junction 403a shown in FIG. 4 so as to direct the reflected light along the Z axis in order to achieve this aspect of the configuration shown. Similarly, the mirror galvanometer 283 shown in FIG. 1 could be positioned at a junction 403b so as to direct the light along the X axis towards the OCT device 281. Each of the fixation display 251, the wavefront sensor 271 and the retinal camera 261 is also associated with a respective junction at which a suitably oriented partial mirror is positioned so as to allow light to travel between the respective device and the first eyepiece 201 along the first optical path defined by the first enclosure 403.

The system 400 may also includes a processing unit 401. The processing unit 401 is in communication with the first ophthalmic devices and the second ophthalmic devices described above. Each ophthalmic device may be controlled by and/or output data to the processing unit 401. For example, the data produced by the OCT device 281 or the retinal camera 261 may output data to the processing unit 401, which may be processed by the processing unit 401 to provide an analysis of the condition of the patient's eye. The processing unit 401 may control the OCT device 281 to vary the length of the reference arm 287 and adjust the positioning of the mirror galvanometer 283. The processing unit 401 may control the fixation display 251, e.g. to vary its position, the brightness of the target presented or the apparent depth of the target as required by the particular measurement being performed. The processing unit 401 is also in communication with the visual field test tool, for example so as to control the display or illumination of an Amsler grid by an associated light source, to control the production of visual stimuli during a Humphrey perimetry test, or to control the pupil tracking camera 309 and illuminating source 307. The processing unit 401 may be in communication with other hardware not shown, for example a display suitable for presenting information to a user of the system 400 or an input device (e.g. a keyboard or touch screen) suitable for receiving input from the user so as to configure the tests or measurements to be performed by the system. In other embodiments the processing unit 401 could be arranged outside of the housing 402, e.g. being in wireless communication with the first and second ophthalmic devices.

FIGS. 4(b) to 4(d) each show orthographic views of the system 400 of FIG. 4(a), with the orientation of each view being indicated by the axes shown.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claims is:

1. An ophthalmic system for providing measurements relating to plural characteristics of a user's eye processed by a common processing device, the system comprising:
a first eyepiece configured to define a first subject region within a field of view of the first eyepiece, the first subject region being configured such that light may travel between the first subject region and one or more first ophthalmic devices through the first eyepiece, each first ophthalmic device being configured to receive light from the first subject region and/or output light to be directed to the first subject region, wherein the first eyepiece and the one or more first ophthalmic devices are arranged such that the light received and/or output by each first ophthalmic device travels between the respective first ophthalmic device and the first eyepiece along a first common path defined by one or more first optical components;
a second eyepiece configured to define a second subject region within a field of view of the second eyepiece, the second subject region being configured such that light may travel between the second subject region and one or more second ophthalmic devices through the second eyepiece, each second ophthalmic device being configured to receive light from the second subject region and/or output light to be directed to the second subject region, wherein the second eyepiece and the one or more second ophthalmic devices are arranged such that the light received and/or output by each second ophthalmic device travels between the respective second ophthalmic device and the second eyepiece along a second common path defined by one or more second optical components;
wherein the second subject region extends across a greater angle in the field of view of the second eyepiece than does the first subject region in the field of view of the first eyepiece, and wherein each of the first and second ophthalmic devices is in communication with the common processing device so as to be controllable by the common processing device and/or output measurements to the common processing device.

2. The ophthalmic system of claim 1, wherein the first and second eyepieces are configured such that the first and second subject regions do not intersect or overlap one another.

3. The ophthalmic system of claim 1, wherein each first and/or second optical component comprises one or more mirrors and/or lenses arranged to direct light along the first or second optical path respectively.

4. The ophthalmic system of claim 1, wherein the one or more first ophthalmic devices comprise one or more of an optical coherence tomography device, a retinal camera and a wavefront sensor.

5. The ophthalmic system of claim 1, wherein the one or more second ophthalmic devices comprise a visual field test tool, preferably an Amsler grid or a display suitable for performing a Humphrey perimetry test.

6. The ophthalmic system of claim 1, wherein the one or more first ophthalmic devices and/or the one or more second ophthalmic devices comprise a fixation display configured to produce a target visually perceptible from the subject region of the respective eyepiece such that a patient at the respective subject region may focus his vision on the target.

7. The ophthalmic system of claim 1, wherein the first and second eyepieces, each of the first and second ophthalmic devices and the processing device are contained by a common housing.

8. The ophthalmic system of claim 1, further comprising a first enclosure configured to enclose the first common path and/or one or more of the first ophthalmic devices; and/or a second enclosure configured to enclose the second common path and/or one or more second ophthalmic devices.

9. The ophthalmic system of claim 8, wherein one or both of the first enclosure and the second enclosure is configured so as to prevent light not transmitted through the respective first or second eyepiece from entering the respective first or second common path.
